(19) United States
(12) Patent Application Publication
(10) Pub. No.: US 2009/0312380 A1
(43) Pub. Date: Dec. 17, 2009

(54) NOVEL COMPOSITIONS AND METHODS

(76) Inventor: Axel Becker, Darmstadt (DE)

Correspondence Address: VENABLE LLP, P.O. BOX 34385, WASHINGTON, DC, 20043-9998, US

(21) Appl. No.: 12/439,269

(22) PCT Filed: Aug. 31, 2007
(86) PCT No.: PCT/GB2007/050519

§ 371 Date: Aug. 6, 2009

(30) Foreign Application Priority Data
Aug. 31, 2006 (GB) 0617171.4

(51) Int. Cl. A61K 31/4184; A61K 31/167; C07D 235/02; C07C 237/00

(52) U.S. Cl. 514/381; 514/630; 548/300.7; 564/220

(57) ABSTRACT

This invention relates to active pharmaceutical ingredients (APIs) with specific water surface areas, to pharmaceutical compositions comprising said APIs, to processes for preparing such compositions, and to methods for determining the water surface areas of substances such as APIs and other particles.

### BACKGROUND OF THE INVENTION

This invention relates to active pharmaceutical ingredients (APIs) with specific water surface areas, to pharmaceutical compositions comprising said APIs, to processes for preparing such compositions, and to methods for determining the water surface areas of substances such as APIs and other particles.

It is standard practice to test and characterise the physiochemical and biological properties of all batches of APIs destined for use in the manufacture of a drug product. Properties such as solubility, water content, particle size, crystal properties, biological activity and permeability are routinely tested and a profile of the API is built up from this data. Such tests are outlined in the ICH Q6A Specifications: Test Procedures and Acceptance Criteria for New Drug Substances and New Drug Products: Chemical Substances. It is essential to ensure that the dissolution kinetics, which are an indication of the bioavailability of an API, are consistent between batches. Indeed, in order to obtain approval to place pharmaceutical products on the market, there must be demonstrable consistency in the dissolution and consequent bioavailability of different batches of the product.

Traditionally, the standard tests employed measure the bulk properties of an API in order to provide characterisation data. Such tests may, for example, include crystalline characterisation and particle size determination. However, even with extensive data from these type of tests, it has been found that there can be variability in the dissolution and/or bioavailability profile of different batches of the same API. Traditional thinking would suggest that APIs of the same polymorphic form and having the same median particle size should show comparable dissolution kinetics. However, the inventors have found on a number of occasions that some batches of particular APIs would pass dissolution testing, but others would unexpectedly fail the same tests. This indicates that the dissolution profiles were different, even though all the batches were manufactured according to the same strict specification and displayed the same characterisation data such as particle size, crystalline form and other physiochemical properties. Consequently, the failed batches could not be used for the preparation of commercial pharmaceutical compositions. In addition, when there are inconsistent dissolution results for APIs having the same crystalline structure and other bulk properties, the manufacturer usually has to discard the batches and there may even be a call for auditing of the testing process and/or the manufacturing to ensure the difference is not due to testing and/or manufacturing procedures. All of this is very inefficient and expensive and ideally should be minimised or avoided.

It would therefore be advantageous to provide APIs wherein the dissolution profile of the particular API has been accurately and predictably characterised. Further there is a need in the art for APIs and pharmaceutical compositions comprising them to be manufactured to certain specifications to ensure a reliable and consistent dissolution profile and bioavailability, thus providing pharmaceutical compositions wherein the bioavailability of the API can be accurately predicted for the safety of the patient. There is also a need in the art for pharmaceutical compositions comprising APIs that have consistent dissolution profiles and improved dissolution kinetics. There is also a need for a testing system to accurately characterise the dissolution profile of an API.

### SUMMARY OF THE INVENTION

The inventors have noted that even when bulk properties, such as the median particle size or crystalline form, of different batches of APIs are characterised and the data is comparable in all cases, there can still be differences in the dissolution profiles of the different batches, i.e. some batches show the expected dissolution rate and some batches show a lesser or higher dissolution rate. Consequently, any pharmaceutical compositions prepared from the APIs are likely to show inconsistent dissolution profiles and different bioavailabilities with the incumbent problems mentioned above. Surprisingly, the inventors have found that the water surface area of an API has an effect on the dissolution kinetics of an API. The inventors have also found that, surprisingly, determining the water surface area of an API can be essential in selecting batches of API that can be used to prepare pharmaceutical compositions having consistent and reliable dissolution profiles. Such pharmaceutical compositions can overcome the problems outlined above. Therefore the present invention provides APIs for use in preparing pharmaceutical compositions that have consistent dissolution profiles and consistent bioavailability.

The present invention is also directed to preparing APIs having optimal water surface area values.

Accordingly, a first aspect of the present invention provides a process for the preparation of a pharmaceutical composition comprising one or more active pharmaceutical ingredient(s) [API(s)] and one or more pharmaceutically acceptable excipient(s), comprising the steps of:
(i) selecting the or each API according to a predetermined water surface area, and
(ii) formulating the or each API with the pharmaceutically acceptable excipient(s).

In a preferred embodiment of the process according to the first aspect of the present invention, the water surface area of the or each API is determined prior to step (i).

The selection of the API is based on predetermined or optimized water surface area values. Optimized water surface area values can be defined as that range of water surface area values that provides for a consistent or uniform dissolution profile when other parameters are comparable. For example, in one embodiment, the optimized water surface area of irbesartan is equal to or greater than about 5 m²/g.

Without wishing to be bound by theory, it is thought that the extent of the water surface area of a particle shows a correlation to the surface hydrophilicity and consequently affects the dissolution behaviour of the particle. An increased surface hydrophilicity is thought to relate to an increased wettability characteristic, thus allowing for compounds according to the invention to show improved dissolution characteristics. Compounds with increased water surface area properties have improved dissolution characteristics. A further advantageous consequence is that batches of API, manufactured to a specification that includes a water surface area according to the invention, allow for reliable prediction of dissolution profiles such that said batches do not need to be discarded due to poor dissolution kinetics of the API. A further advantage envisaged is that when multiple pilot-scale batches are made for testing, characterisation of the water surface area will aid in determining which batches should be used for preparing compositions for market. The opposite scenario would be using all batches with equivalent bulk properties and testing the resultant compositions. It can be predicted that those compositions comprising API having unfavourable water surface area values would not be passed and would subsequently be discarded at great cost.

In one embodiment of the process according to the first aspect of the present invention, the API is poorly soluble in an aqueous medium. Preferably the API is irbesartan, more preferably the water surface area of irbesartan is equal to or greater than about 5 m²/g.

In an alternative embodiment of the process according to the first aspect of the present invention, the API is poorly soluble in a non-aqueous medium, preferably a polar non-aqueous medium.

A second aspect of the present invention provides a pharmaceutical composition comprising an API having a predetermined water surface area. Preferably the API is a solid, such as a particulate or a powder.

In one embodiment according to the second aspect of the present invention, the composition is a solid composition, preferably in the form of a tablet, a capsule, or a dry powder. Advantageously the API is irbesartan, which in favoured embodiments has a water surface area equal to or greater than about 5 m²/g.

In another embodiment according to the second aspect of the present invention, the composition is a liquid composition, such as a suspension or emulsion, which preferably is a parenteral composition or alternatively an oral liquid.

In a further embodiment according to the second aspect of the present invention, there is provided a topical composition. Preferably the composition is in the form of a gel, an ointment, a balm, a nasal spray, eye drops, or a cream.

In a still further embodiment according to the second aspect of the present invention, the composition is formulated for inhalation. In preferable embodiments, the composition is formulated for use in a dry powder inhaler (DPI), a metered dose inhaler (MDI), or a nebule. Preferably the composition is formulated for use in a dry powder inhaler (DPI). Most preferably, the API is one or a combination of formoterol, salmeterol, fluticasone, budesonide or any pharmaceutically acceptable salts thereof. In further preferred embodiments, the API is one or more of salmeterol xinafoate, fluticasone propionate, budesonide, or formoterol fumarate. In a preferred embodiment, the composition further comprises lactose.

The surface hydrophilicity of API samples for inhalers will have an impact on the interaction strength of the API fine particles with the inhaler carrier materials (such as lactose monohydrate), hence governing the available Fine Particle Fraction (FPF) and Fine Particle Dose (FPD) of API material which will be dispersed (i.e. de-aggregated) from the carrier material upon application of the inhaler device, and hence directly impacting on the amount of API material potentially available in the lungs.

A third aspect of the present invention provides a process for the preparation of pharmaceutical compositions which possess uniform dissolution and/or bioavailability, comprising the steps of:
(i) taking a sample from the or each of one or more batches of active pharmaceutical ingredient (API),
(ii) measuring the water surface area of the or each sample,
(iii) selecting the batch(es) of API to be used in the preparation of pharmaceutical compositions based on the measured water surface area values, and
(iv) formulating said API from the selected batch(es) into pharmaceutical compositions.

The bioavailability of compositions is considered ‘uniform’ or ‘equivalent’, if from composition to composition the bioavailability typically varies no more than between about 0.8 and about 1.25. Any dissolution rates of compositions that lead to uniform or equivalent bioavailability are considered ‘uniform’.

In a preferred embodiment of the process according to the third aspect of the present invention, the water surface area of each sample is measured using gravimetric vapour sorption (GVS).

In another embodiment of the process according to the third aspect of the present invention, the batches are selected by comparing the measured water surface area values with predetermined water surface area values and selecting those batches having water surface area values within the predetermined range.

In yet another embodiment of the process according to the third aspect of the present invention, the API is poorly soluble in an aqueous medium or alternatively the API is poorly soluble in a non-aqueous medium, preferably a polar non-aqueous medium such as ethanol or methanol.

In one embodiment of the process according to the third aspect of the present invention, the composition is a solid composition, preferably in the form of a tablet, a capsule, or a dry powder. Advantageously the API is irbesartan, which in favoured embodiments has a water surface area equal to or greater than about 5 m²/g.

In another embodiment of the process according to the third aspect of the present invention, the composition is a liquid composition, preferably a suspension or emulsion, which preferably is a parenteral composition or alternatively an oral liquid.

In a further embodiment of the process according to the third aspect of the present invention, there is provided a topical composition. Preferably the composition is in the form of a gel, an ointment, a balm, a nasal spray, eye drops, or a cream.

In a still further embodiment of the process according to the third aspect of the present invention, there is provided a composition formulated for inhalation. In preferable embodiments, the composition is formulated for use in a dry powder inhaler (DPI), a metered dose inhaler (MDI), or a nebule. Preferably the composition is formulated for use in a dry powder inhaler (DPI). Most preferably, the API is one or a combination of formoterol, salmeterol, fluticasone, budesonide or any pharmaceutically acceptable salts thereof. In further preferred embodiments, the API is one or more of salmeterol xinafoate, fluticasone propionate, or hydrated formoterol fumarate. In a preferred embodiment, the composition further comprises lactose.

A fourth aspect of the present invention provides a method for determining the water surface area of an API, comprising the steps of:

(i) measuring water vapour sorption isotherms of a sample of the API, and
(ii) applying a model for determining the water surface area.

In particularly preferred embodiments, the isotherms are measured using gravimetric vapour sorption (GVS). A preferred model for determining water surface area is the Excess Surface Work (ESW) model or, in alternative embodiments, the Brunauer, Emmet and Teller (BET) model.

In preferred embodiments, the API is irbesartan, which most favourably has a water surface area equal to or greater than about 5 m²/g.

A fifth aspect of the present invention provides a method for determining the water surface area of a particle, comprising the steps of:

(i) measuring water vapour sorption isotherms of a sample of the particle, and
(ii) applying a model for determining the water surface area.

In particularly preferred embodiments, the isotherms are measured using gravimetric vapour sorption (GVS). A preferred model for determining water surface area is the Excess Surface Work (ESW) model or, in alternative embodiments, the Brunauer, Emmet and Teller (BET) model.

In preferred embodiments, the API is irbesartan, which most favourably has a water surface area equal to or greater than about 5 m²/g.

A sixth aspect of the present invention provides irbesartan with a water surface area equal to or greater than about 5 m²/g, preferably equal to or greater than about 5.5 m²/g, preferably equal to or greater than about 6 m²/g, preferably equal to or greater than about 6.5 m²/g, preferably equal to or greater than about 7 m²/g.

A seventh aspect of the present invention provides an API for use in an inhaler, said API having a water surface area that allows for optimized adherence to a support suitable for use in the inhaler. In preferred embodiments, the API is one or more of formoterol, salmeterol, fluticasone, budesonide or any pharmaceutically acceptable salts thereof. In further preferred embodiments, the API is one or more of salmeterol xinafoate, fluticasone propionate, budesonide, or formoterol fumarate. Preferably the inhaler is a dry powder inhaler (DPI). In further embodiments the support is a particulate support, which most preferably is lactose.

In the case of APIs for use in an inhaler, optimized water surface area values can be defined as that range of water surface area values that provides for a consistent or uniform adherence to a support suitable for use in the inhaler when other parameters are comparable. The adherence of API batches is considered ‘uniform’, if from batch to batch the adherence typically varies no more than between about 0.8 and about 1.25. A uniform adherence is an optimized adherence.

An eighth aspect of the present invention provides a process for assessing the surface hydrophilicity of an API or a particle, said process comprising combining water surface area (WSA) and specific surface area (SSA) (BET nitrogen).

A combination according to the eighth aspect of the present invention may be performed for instance by obtaining the ratio of the WSA:SSA, or by taking as the relevant value the lesser of the two values. Other combinations, such as adding the values, multiplying them, or entering them into more complex mathematical formulae, with or without additional variables, are also envisaged.

It is preferred that where the WSA and SSA are combined, both the WSA and SSA have been calculated using the same mathematical model, preferably the BET model.

A ninth aspect of the present invention provides a process comprising measuring the water surface area of a substance. Preferably said measuring comprises the steps of:
(i) measuring water vapour sorption isotherms of a sample of the substance, and
(ii) applying a model for determining water surface area.

In any embodiment of the present invention that involves measuring water vapour sorption isotherms of a sample, the isotherms may be measured using any technique known to those skilled in the art, such as gravimetric vapour sorption (GVS), or a non-gravimetric technique such as a water partial pressure monitoring sorption system.

Suitable models for determining the water surface area include the Excess Surface Work (ESW) model, the Brunauer, Emmet and Teller (BET) model, the Langmuir isotherm, the Temkin isotherm, and the Freundlich isotherm. Preferred models are the Excess Surface Work (ESW) model, and the Brunauer, Emmet and Teller (BET) model.

In one embodiment, the substance is poorly soluble in an aqueous medium. In an alternative embodiment, the substance is poorly soluble in a non-aqueous medium, preferably a polar non-aqueous medium. The substance may be a solid, preferably in particulate or powder form.

As used herein, a solid in particulate form relates to a solid wherein the median particle diameter is less than 2 mm, preferably less than 1 mm, more preferably less than 0.5 mm.

As used herein, a solid in powder form relates to a solid wherein the median particle diameter is less than 200 μm, preferably less than 100 μm or 50 m, most preferably less than 10 μm.

As used herein, the term ‘poorly soluble’ includes sparingly soluble, slightly soluble, very slightly soluble, and practically insoluble. In a preferred embodiment, the term ‘poorly soluble’ only includes slightly soluble, very slightly soluble, and practically insoluble. In another preferred embodiment, the term ‘poorly soluble’ only includes very slightly soluble and practically insoluble. These terms are defined in the European and US Pharmacopeia as follows:

| | volume of solvent in millilitres per gram of |
|---|---|
| | solute (EP) or parts of solvent required for |
| | 1 part of solute (USP) |
| sparingly soluble | from 30 to 100 |
| slightly soluble | from 100 to 1,000 |
| very slightly soluble | from 1,000 to 10,000 |
| practically insoluble | from 10,000 and over |

Polar non-aqueous mediums include protic polar mediums such as alcohols including methanol and ethanol, and carboxylic acids including acetic acid. Polar non-aqueous mediums also include dipolar aprotic mediums such as dimethyl sulphoxide, dimethyl formamide and acetonitrile.

In a preferred embodiment, the substance is an active pharmaceutical ingredient (API). APIs suitable for use in relation to any aspect of the present invention include the following sparingly soluble to insoluble solid APIs: acenocoumarol, acetaminophen, acetazolamide, acetohexamide, acetyl digitoxin, acyclovir, adenine, albendazole, albuterol, allantoin, allopurinol, alprazolam, altretamine, amikacin, amiloride hydrochloride, aminobenzoic acid, aminoglutethimide, aminohippuric acid, aminosalicylic acid, amodiaquine, amoxapine, amoxicillin, amphotericin B, ampicillin, anileridine, anthralin, apomorphine hydrochloride, apraclonidine hydrochloride, arsanilic acid, aspirin, atenolol, atovaquone, atropine, azathioprine, bacitracin zinc, baclofen, barium sulphate, beclomethasone dipropionate, bendroflumethiazide, benzocaine, betacarotene, betamethasone, betamethasone acetate, betamethasone benzoate, betamethasone dipropionate, betamethasone valerate, biotin, biperiden, biperiden hydrochloride, bisacodyl, bismuth citrate, bismuth subcarbonate, bismuth subgallate, bismuth subnitrate, bismuth subsalicylate, brinzolamide, bumetanide, busulfan, butabarbital, butalbital, butamben, butoconazole nitrate, butorphanol tartrate, caffeine, capsaicin, carbamazepine, carbidopa, carisoprodol, cefaclor, cefadroxil, cefazolin, cefixime, cefinenoxime hydrochloride, ceforanide, cefpodoxime proxetil, ceftazidime, cefuroxime axetil, cephalexin, cephapirin benzathine, cephradine, chlorambucil, chloramphenicol, chloramphenicol palmitate, chlordiazepoxide, chlorhexidine hydrochloride, chlorobutanol, chloroquine, chlorothiazide, chloroxylenol, chlorpromazine, chlorpropamide, chlortetracycline hydrochloride, chlorthalidone, chlorzoxazone, cholecalciferol, ciclopirox, ciclopirox olamine, cimetidine, cinoxacin, ciprofloxacin hydrochloride, clarithromycin, clemastine fumarate, clioquinol, clobetasol propionate, clofazimine, clofibrate, clomiphene citrate, clonazepam, clorsulon, clotrimazole, cloxacillin benzathine, clozapine, cocaine, codeine, codeine sulphate, cortisone acetate, cyanocobalamin, cyclandelate, cyclizine hydrochloride, cyclosporine, cyproheptadine hydrochloride, dactinomycin, danazol, dapsone, dehydrocholic acid, demeclocycline, demeclocycline hydrochloride, desoximetasone, desoxycorticosterone acetate, dexamethasone, dexamethasone acetate, dextromethorphan, dextromethorphan hydrobromide, diatrizoic acid, diazepam, diazoxide, dibucaine, diclofenac sodium, dienestrol, diethylstilbestrol, diethylstilbestrol diphosphate, diflorasone diacetate, diflunisal, digitoxin, digoxin, dihydroergotamine mesylate, dihydrotachysterol, diloxanide furoate, dimenhydrinate, dioxybenzone, diphenoxylate hydrochloride, dipyridamole, dirithromycin, disulfiram, dobutamine hydrochloride, docusate calcium, docusate potassium, docusate sodium, doxapram hydrochloride, doxycycline, droperidol, dyclonine hydrochloride, dydrogesterone, econazole nitrate, enalapril maleate, enalaprilat, enflurane, epinephrine, ergocalciferol, ergoloid mesylates, ergonovine maleate, ergotamine tartrate, erythromycin, erythromycin estolate, erythromycin ethylsuccinate, erythromycin stearate, estradiol, estradiol cypionate, estradiol valerate, estriol, estrone, estropipate, ethacrynic acid, ethinyl estradiol, ethionamide, ethopabate, ethotoin, ethynodiol diacetate, etoposide, famotidine, felodipine, fenbendazole, fenoprofen calcium, fentanyl citrate, finasteride, fluconazole, flucytosine, fludarabine phosphate, fludrocortisone acetate, flumazenil, flumethasone pivalate, flunisolide, fluocinolone acetonide, fluocinonide, fluorescein, fluorometholone, fluorouracil, fluoxetine hydrochloride, fluoxymesterone, fluphenazine enanthate, flurandrenolide, flurbiprofen, fluroxene, flutamide, fluvoxamine maleate, folic acid, furazolidone, furosemide, gemfibrozil, gentian violet, glimepiride, gramicidin, griseofulvin, guaifenesin, guanabenz acetate, halazone, halcinonide, haloperidol, hexachlorophene, hydrochlorothiazide, hydrocortisone, hydrocortisone acetate, hydrocortisone butyrate, hydroflumethiazide, hydroxocobalamin, hydroxyprogesterone caproate, hydroxyzine pamoate, hyoscyamine, ibuprofen, idarubicin hydrochloride, idoxuridine, imipenem, inamrinone, indapamide, indigotindisulfonate sodium, indomethacin, inulin, iodine, iodipamide, iodoform, iodoquinol, iopanoic acid, iophendylate, iothalamic acid, irbesartan, isopropamide iodide, isosorbide dinitrate, isotretinoin, isoxsuprine hydrochloride, ivermectin, lansoprazole, letrozole, levodopa, levonordefrin, levonorgestrel, levorphanol tartrate, levothyroxine sodium, lidocaine, lindane, liothyronine sodium, loperamide hydrochloride, loratadine, lorazepam, lovastatin, magaldrate, maprotiline hydrochloride, mazindol, mebendazole, meclizine hydrochloride, medroxyprogesterone acetate, mefanamic acid, mefloquine hydrochloride, megestrol acetate, melengestrol acetate, melphalan, menadione, mephobarbital, meprobamate, mercaptopurine, ammoniated mercury, meropenem, mesalamine, mestranol, methacycline hydrochloride, methazolamide, methocarbamol, methohexital, methotrexate, methotrimeprazine, methozsalen, methsuximide, methyclothiazide, methyl salicylate, methyldopa, methylergonovine maleate, methylprednisolone, methylprednisolone acetate, methylprednisolone hemisuccinate, methyltestosterone, methysergide maleate, metronidazole, metronidazole benzoate, metyrapone, mibolerone, miconazole, miconazole nitrate, milrinone, minocycline hydrochloride, minoxidil, mirtazapine, mitomycin, mitotane, mitoxantrone hydrochloride, monensin sodium, mupirocin, nabumetone, nadolol, nalidixic acid, nandrolone decanoate, naproxen, natamycin, nevirapine, niacin, nifedipine, nimodipine, nitrofurantoin, nitrofurazone, nitromersol, nizatidine, norethindrone, norethindrone acetate, norethynodrel, norfloxacin, norgestimate, norgestrel, nortriptyline hydrochloride, noscapine, novobiocin calcium, nystatin, ofloxacin, omeprazole, ondansetron, ondansetron hydrochloride, orphenadrine citrate, oxandrolone, oxazepam, oxfendazole, oxybenzone, oxymetholone, oxytetracycline, oxytetracycline calcium, paclitaxel, padimate O, papaverine hydrochloride, parachlorophenol, paramethasone acetate, paricalcitol, paroxetine hydrochloride, penicillin G benzathine, penicillin G procaine, penicillin G sodium, penicillin V, penicillin V benzathine, pentazocine, pentazocine hydrochloride, pentobarbital, pergolide mesylate, perphenazine, phenazopyridine hydrochloride, phenindamine tartrate, phenobarbital, phenolsulfonphthalein, phensuximide, phenylbenzimidazole sulfonic acid, phenylbutazone, phenyltoloxamine citrate, phenytoin, physostigmine, physostigmine salicyclate, piminodine esylate, pimozide, pindolol, piperacillin, piroxicam, plicamycin, praziquantel, prazosin hydrochloride, prednisolone, prednisolone acetate, prednisolone hemisuccinate, prednisolone tebutate, prednisone, prilocalne, primidone, probucol, probenecid, prochlorperazine, prochlorperazine maleate, procyclidine hydrochloride, progesterone, propafenone hydrochloride, propoxyphene napsylate, propyliodone, propylparaben, propylthiouracil, pyrantel pamoate, pyrazinamide, pyrimethamine, pyrvinium pamoate, quinidine sulphate, quinine sulphate, racepinephrine, ramipril, reserpine, riboflavin, riboflavin 5′-phosphate sodium, rifabutin, rifampin, roxarsone, salicyclamide, salicyclic acid, secobarbital, simvastatin, spironolactone, stanozolol, storax, sulconazole nitrate, sulfabenzamide, sulfacetamide, sulfadiazine, sulfadimethoxine, sulfaethidole, sulfamethazine, sulfamethizole, sulfamethoxazole, sulfapyridine, sulfasalazine, sulfathiazole, sulfinpyrazone, sulfisoxazole, sulfisoxazole acetyl, sulindac, sumatriptan, suprofen, tamoxifen citrate, temazepam, terpin hydrate, testolactone, testosterone, testosterone cypionate, testosterone enanthate, testosterone propionate, tetracaine, tetracycline, thalidomide, theophylline, thiabendazole, thiacetarsamide, thiamine mononitrate, thiethylperazine maleate, thioguanine, thioridazine, thiostrepton, thiothixene, thymol, tiagabine hydrochloride, tilmicosin, tinidazole, tolazamide, tolbutamide, tolcapone, tolnaftate, torsemide, trazodone hydrochloride, tretinoin, triamcinolone, triamcinolone acetonide, triamcinolone diacetate, triamcinolone hexacetonide, triamterene, triazolam, trichlormethiazide, triclosan, trihexyphenidyl hydrochloride, trimethoprim, trioxsalen, troleandomycin, tropicamide, tylosin, ubidecarenone, undecylenic acid, ursodiol, valrubicin, vecuronium bromide, vidarabine, vitamin A, xylazine, xylazine hydrochloride, xylometazoline hydrochloride, yohimbine hydrochloride, and zidovudine.

APIs suitable for use in relation to any aspect of the present invention include the following very slightly soluble to insoluble solid APIs: acenocoumarol, acetazolamide, acetohexamide, acetyl digitoxin, adenine, albendazole, allopurinol, alprazolam, altretamine, aminoglutethimide, amodiaquine, amoxapine, amphotericin B, anileridine, anthralin, atovaquone, azathioprine, barium sulphate, beclomethasone dipropionate, bendroflumethiazide, benzocaine, betacarotene, betamethasone, betamethasone acetate, betamethasone benzoate, betamethasone dipropionate, betamethasone valerate, biotin, biperiden, bisacodyl, bismuth citrate, bismuth subcarbonate, bismuth subgallate, bismuth subnitrate, bismuth subsalicylate, brinzolamide, busulfan, butabarbital, butamben, butoconazole nitrate, capsaicin, carbamazepine, carisoprodol, cefixime, cefinenoxime hydrochloride, ceforanide, cefpodoxime proxetil, cefuroxime axetil, cephapirin benzathine, chlorambucil, chloramphenicol palmitate, chlordiazepoxide, chloroquine, chlorothiazide, chloroxylenol, chlorpromazine, chlorpropamide, chlorthalidone, cholecalciferol, cinoxacin, clarithromycin, clemastine fumarate, clioquinol, clobetasol propionate, clofazimine, clofibrate, clonazepam, clotrimazole, clozapine, cortisone acetate, cyclandelate, cyclosporine, danazol, dapsone, dehydrocholic acid, desoximetasone, desoxycorticosterone acetate, dexamethasone, dexamethasone acetate, dextromethorphan, diatrizoic acid, diazepam, diazoxide, dienestrol, diethylstilbestrol, diflorasone diacetate, diflunisal, digitoxin, digoxin, dihydrotachysterol, diloxanide furoate, dioxybenzone, dirithromycin, disulfiram, docusate calcium, doxycycline, droperidol, dydrogesterone, econazole nitrate, epinephrine, ergocalciferol, ergotamine tartrate, erythromycin estolate, erythromycin ethylsuccinate, erythromycin stearate, estradiol, estradiol cypionate, estradiol valerate, estriol, estrone, estropipate, ethacrynic acid, ethinyl estradiol, ethopabate, ethotoin, ethynodiol diacetate, etoposide, famotidine, felodipine, fenbendazole, finasteride, fludrocortisone acetate, flumazenil, flumethasone pivalate, flunisolide, fluocinolone acetonide, fluocinonide, fluorescein, fluorometholone, fluoxymesterone, fluphenazine enanthate, flurandrenolide, flurbiprofen, flutamide, folic acid, furazolidone, furosemide, gemfibrozil, glimepiride, gramicidin, griseofulvin, halazone, halcinonide, haloperidol, hexachlorophene, hydrocortisone, hydrocortisone acetate, hydrocortisone butyrate, hydroflumethiazide, hydroxyprogesterone caproate, hydroxyzine pamoate, ibuprofen, inamrinone, indapamide, indomethacin, iodine, iodipamide, iodoform, iodoquinol, iopanoic acid, iophendylate, irbesartan, isosorbide dinitrate, isotretinoin, ivermectin, lansoprazole, letrozole, levonordefrin, levonorgestrel, levothyroxine sodium, lidocaine, lindane, liothyronine sodium, loratadine, lorazepam, lovastatin, magaldrate, mazindol, mebendazole, meclizine hydrochloride, medroxyprogesterone acetate, mefanamic acid, mefloquine hydrochloride, megestrol acetate, melengestrol acetate, melphalan, menadione, mercaptopurine, ammoniated mercury, mestranol, methazolamide, methohexital, methotrexate, methotrimeprazine, methozsalen, methyclothiazide, methylprednisolone, methylprednisolone acetate, methylprednisolone hemisuccinate, methyltestosterone, metronidazole benzoate, mibolerone, miconazole, miconazole nitrate, milrinone, mirtazapine, mitotane, mupirocin, nabumetone, nalidixic acid, nandrolone decanoate, naproxen, natamycin, nevirapine, nifedipine, nimodipine, nitrofurantoin, nitrofurazone, nitromersol, norethindrone, norethindrone acetate, norethynodrel, norgestimate, norgestrel, noscapine, nystatin, omeprazole, oxandrolone, oxazepam, oxfendazole, oxybenzone, oxymetholone, oxytetracycline, oxytetracycline calcium, paclitaxel, padimate O, paramethasone acetate, paricalcitol, penicillin G benzathine, penicillin V, penicillin V benzathine, pentazocine, pentobarbital, perphenazine, phenobarbital, phenolsulfonphthalein, phenylbenzimidazole sulfonic acid, phenylbutazone, phenytoin, piminodine esylate, pimozide, pindolol, piperacillin, piroxicam, praziquantel, prednisolone, prednisolone acetate, prednisolone hemisuccinate, prednisolone tebutate, prednisone, primidone, probucol, probenecid, prochlorperazine, prochlorperazine maleate, progesterone, propoxyphene napsylate, propyliodone, propylparaben, pyrantel pamoate, pyrimethamine, pyrvinium pamoate, racepinephrine, reserpine, riboflavin, rifabutin, rifampin, secobarbital, simvastatin, spironolactone, stanozolol, storax, sulconazole nitrate, sulfabenzamide, sulfadiazine, sulfadimethoxine, sulfaethidole, sulfamethazine, sulfamethizole, sulfamethoxazole, sulfapyridine, sulfasalazine, sulfathiazole, sulfinpyrazone, sulfisoxazole, sulfisoxazole acetyl, sulindac, sumatriptan, tamoxifen citrate, temazepam, testosterone, testosterone cypionate, testosterone enanthate, testosterone propionate, tetracaine, tetracycline, thiabendazole, thiethylperazine maleate, thioguanine, thioridazine, thiostrepton, thiothixene, thymol, tinidazole, tolazamide, tolbutamide, tolcapone, tolnaftate, torsemide, tretinoin, triamcinolone, triamcinolone acetonide, triamcinolone diacetate, triamcinolone hexacetonide, triamterene, triazolam, trichlormethiazide, triclosan, trihexyphenidyl hydrochloride, trimethoprim, trioxsalen, ubidecarenone, undecylenic acid, ursodiol, valrubicin, vidarabine, and vitamin A.

In a preferred embodiment, the API is irbesartan, formoterol, salmeterol, fluticasone, or budesonide, more preferably irbesartan.

The present invention can also be applied to other pharmaceutically acceptable substances. Therefore in another embodiment, the substance is a pharmaceutically acceptable excipient. Pharmaceutically acceptable excipients suitable for use in relation to any aspect of the present invention include the following sparingly soluble to insoluble solid excipients: adipic acid, agar, alginic acid, dried aluminum hydroxide gel, aluminum monostearate, ammonia methacrylate copolymer, ascorbyl palmitate, aspartame, aspartame acesulfame, aspartic acid, activated attapulgite, colloidal activated attapulgite, bentonite, purified bentonite, benzoic acid, hydrous benzoyl peroxide, betadex, butylated hydroxyanisole, butylated hydroxytoluene, butylparaben, calamine, calcium carbonate, calcium citrate, calcium gluconate, calcium hydroxide, dibasic calcium phosphate, tribasic calcium phosphate, calcium polycarbophil, calcium saccharate, calcium silicate, calcium stearate, calcium sulphate, calcium undecylenate, camphor, candelilla wax, carbomer copolymer, carbomer interpolymer, carboxymethylcellulose calcium, carboxymethylcellulose sodium, hydrogenated castor oil, cellaburate, cellacefate, cellulose acetate, microcrystalline cellulose, powdered cellulose, cellulose sodium phosphate, cetostearyl alcohol, cetyl alcohol, cetyl esters wax, cetyl palmitate, activated charcoal, chlorocresol, cholesterol, cholestyramine resin, coal tar, cocoa butter, colestipol hydrochloride, hydrogenated cottonseed oil, cresol, croscarmellose sodium, crospovidone, dextrin, diethylene glycol stearates, diethyl phthalate, dihydroxyaluminum aminoacetate, dihydroxyaluminum sodium carbonate, edetic acid, ethyl vanillin, ethylcellulose, ethylene glycol stearates, ethylparaben, hard fat, ferric oxide, ferric sulphate, ferrous fumarate, dried ferrous sulphate, fumaric acid, gelatine, glyceryl behenate, glyceryl distearate, glyceryl monolinoleate, glyceryl monooleate, glyceryl monostearate, guar gum, gutta percha, hydroxypropyl cellulose, hypromellose, hypromellose acetate succinate, hypromellose phthalate, juniper tar, kaolin, lactose monohydrate, lanolin, lanolin alcohols, lecithin, leucine, lime, lithium carbonate, magnesium aluminometasilicate, magnesium aluminosilicate, magnesium aluminum silicate, magnesium carbonate, magnesium hydroxide, magnesium oxide, magnesium phosphate, magnesium silicate, magnesium stearate, magnesium trisilicate, menthol, methyl benzylidene camphor, methylcellulose, methylparaben, monoglyceride citrate, myristic acid, octyldodecanol, palmitic acid, paraffin, synthetic paraffin, pectin, petrolatum, white petrolatum, phenoxyethanol, phenylalanine, phenylmercuric acetate, phenylmercuric nitrate, polacrillin potassium, polycarbophil, polyisobutylene, polyoxyl stearyl ether, polyvinyl acetate phthalate, potassium bitartrate, potassium metaphosphate, propyl gallate, propylene glycol monostearate, pumice, saccharin, selenium sulphide, shellac, dental-type silica, purified siliceous earth, silicon dioxide, colloidal silicon dioxide, soda lime, sodium polystyrene sulphonate, sodium stearate, sodium stearyl fumarate, sorbic acid, sorbitan monopalmitate, sorbitan monostearate, hydrogenated soybean oil, starch, corn starch, potato starch, pregelatinised starch, pregelatinised modified starch, tapioca starch, wheat starch, stearic acid, purified stearic acid, stearyl alcohol, sucrose octaacetate, precipitated sulphur, sublimed sulphur, talc, titanium dioxide, tolu balsam, tyrosine, vanillin, hydrogenated vegetable oil, carnauba wax, emulsifying wax, microcrystalline wax, white wax, yellow wax, wheat bran, zein, zinc oxide, zinc stearate, and zinc undecylenate.

Pharmaceutically acceptable excipients suitable for use in relation to any aspect of the present invention include the following very slightly soluble to insoluble solid excipients: agar, alginic acid, dried aluminum hydroxide gel, aluminum monostearate, ammonia methacrylate copolymer, ascorbyl palmitate, activated attapulgite, colloidal activated attapulgite, bentonite, purified bentonite, butylated hydroxyanisole, butylated hydroxytoluene, butylparaben, calamine, calcium carbonate, dibasic calcium phosphate, tribasic calcium phosphate, calcium polycarbophil, calcium saccharate, calcium silicate, calcium stearate, calcium sulphate, calcium undecylenate, candelilla wax, carbomer copolymer, carbomer interpolymer, carboxymethylcellulose calcium, carboxymethylcellulose sodium, hydrogenated castor oil, cellaburate, cellacefate, cellulose acetate, microcrystalline cellulose, powdered cellulose, cellulose sodium phosphate, cetostearyl alcohol, cetyl alcohol, cetyl esters wax, cetyl palmitate, activated charcoal, cholesterol, cholestyramine resin, cocoa butter, colestipol hydrochloride, hydrogenated cottonseed oil, crospovidone, diethylene glycol stearates, diethyl phthalate, dihydroxyaluminum aminoacetate, dihydroxyaluminum sodium carbonate, edetic acid, ethylcellulose, ethylene glycol stearates, ethylparaben, hard fat, ferric oxide, glyceryl behenate, glyceryl distearate, glyceryl monolinoleate, glyceryl monooleate, glyceryl monostearate, guar gum, gutta percha, hypromellose acetate succinate, hypromellose phthalate, juniper tar, kaolin, lanolin, lanolin alcohols, magnesium aluminometasilicate, magnesium aluminosilicate, magnesium aluminum silicate, magnesium carbonate, magnesium hydroxide, magnesium oxide, magnesium phosphate, magnesium silicate, magnesium stearate, magnesium trisilicate, methyl benzylidene camphor, monoglyceride citrate, myristic acid, octyldodecanol, palmitic acid, paraffin, synthetic paraffin, petrolatum, white petrolatum, phenylmercuric nitrate, polacrillin potassium, polycarbophil, polyisobutylene, polyoxyl stearyl ether, polyvinyl acetate phthalate, potassium metaphosphate, propylene glycol monostearate, pumice, selenium sulphide, shellac, dental-type silica, purified siliceous earth, silicon dioxide, colloidal silicon dioxide, soda lime, sodium polystyrene sulphonate, sodium stearyl fumarate, sorbitan monopalmitate, sorbitan monostearate, hydrogenated soybean oil, starch, corn starch, potato starch, tapioca starch, wheat starch, stearic acid, purified stearic acid, stearyl alcohol, sucrose octaacetate, precipitated sulphur, sublimed sulphur, talc, titanium dioxide, tolu balsam, tyrosine, hydrogenated vegetable oil, carnauba wax, emulsifying wax, microcrystalline wax, white wax, yellow wax, wheat bran, zein, zinc oxide, zinc stearate, and zinc undecylenate.

One embodiment of the process of the ninth aspect of the present invention further comprises the step of comparing the water surface area with a predetermined value or range of values. For example, the water surface area may be used to predict the rate of dissolution of a sample. Accordingly, the sample may then be rejected as having too slow a rate of dissolution, if its water surface area value is below a predetermined value. Alternatively, where a slow-release formulation is required, a sample may be rejected, if its water surface area value is above a predetermined value.

Another embodiment of the process of the ninth aspect of the present invention further comprises the step of using the water surface area to predict another property of the substance, such as the dissolution rate. As explained in relation to the eighth aspect of the present invention, the water surface area of a substance may be used in conjunction with another value, such as the specific surface area of the substance, to predict the other property.

A tenth aspect of the present invention provides a method of manufacturing a substance, said method comprising the process of the ninth aspect of the present invention.

An eleventh aspect of the present invention provides a method of manufacturing a substance, wherein the manufacturing process is performed to meet a predetermined water surface area value.

A twelfth aspect of the present invention provides a method of altering a manufacturing process for a substance, comprising the alteration of a process variable in response to a water surface area value.

### BRIEF DESCRIPTION OF THE DRAWINGS

FIG. 1: XRPD traces of samples A-D.

FIG. 2: BET plot of p/pₛ versus [p/pₛ]/[n(1−p/pₛ)] for sample A.

FIG. 3: ESW plot of Φ/RT versus change in mass for sample A.

FIG. 4: Powder dissolution profiles in 0.1N HCl (37° C.) of samples of irbesartan.

FIG. 5: Powder dissolution profiles in phosphate buffer at pH 7.2 (37° C.) of samples of irbesartan.

### DETAILED DESCRIPTION OF THE INVENTION

As described above, it is standard practice to measure the bulk properties of APIs in order to accurately characterise the parameters of said APIs. Properties of an API such as melting point data, particle size and polymorphism can be measured using a variety of techniques including X-ray powder diffraction (XRPD), infra-red, Raman spectroscopy and differential scanning calorimetry (DSC).

During development of a pharmaceutical product it is generally bulk properties that are taken into account. Therefore different salt forms, crystalline forms and crystalline morphology and the corresponding physiochemical properties such as melting point, particle size and dissolution are all well determined and provide the bulk of the characterising information about an API. It has been found however that measurement of these properties can provide misleading results, sometimes having far-reaching consequences.

The inventors found when testing different batches of API destined to be used for marketed pharmaceutical compositions, that there were large differences in dissolution profiles. In fact some of the batches did not meet the required parameters and had to be discarded. The bulk properties such as particle size and the crystalline form of the API being tested were all very similar, therefore it was very difficult to determine the reasons for the inconsistent dissolution profiles. The inventors found further that, surprisingly, there were differences in the water surface area of the particles of the different batches. The water surface area is a measure of the surface hydrophilicity of a particle. Novel techniques have been developed to determine the water surface area of an API.

Accordingly, there is provided a method for determining the water surface area of an API, comprising the steps of:

(i) measuring water vapour sorption isotherms of a sample of the API, and
(ii) applying a model for determining the water surface area.

In another embodiment of the invention, gravimetric vapour sorption (GVS) was used to determine the water vapour sorption of the samples.

### Gravimetric Vapour Sorption (GVS)

GVS is a method traditionally used to detect quantitative weight changes of samples as a function of the relative humidity (RH). Samples, which can comprise solid-state samples such as powders and tablets, but also liquids, are exposed to a defined humidity profile. The corresponding weight change is continuously recorded via an ultra-microbalance. Defined humidity levels are established by mixing defined ratios of a dry carrier gas, usually nitrogen, and a wet stream, typically a water vapour saturated carrier gas such as nitrogen. The RH level is then controlled by the ratio of the dry:wet stream. To maintain isothermal conditions, the GVS apparatus which comprises a microbalance and humidification unit is set up in an incubator system held at a defined temperature.

The primary information obtained by a typical commercial GVS system is:
Kinetics of water uptake:
The kinetic of the water uptake is a mass vs. time plot at constant RH level. The equilibrium between sample and humidity can be determined from this information.
Sorption isotherms:
The equilibrium weight changes at each humidity are related to the dry mass of the sample (i.e. the equilibrium mass at 0% RH). The equilibrium weight changes as a function of the RH level are the corresponding sorption isotherms of the sample. Typical sorption isotherms comprise adsorption, i.e. increasing RH levels, and desorption, i.e. decreasing RH levels, cycles. Typically, RH ranges from 0-95% RH are covered.

### Typical Use of GVS:

GVS is routinely used as a method to obtain the kinetics and sorption isotherms of water sorption processes that take place at the surface of samples. The results provide information about the sample in terms of:
Hygroscopicity:
The extent of water uptake levels at defined RH levels are used to assess the hygroscopicity of materials. From measuring water uptake levels at different RH levels, potential critical RH levels can be assessed where the investigated compound exceeds a certain specified water uptake threshold. An example of critical RH levels is the deliquescence point, which is a compound-specific value. If a sample exhibits a deliquescence point, there will be a very strong water uptake at RH levels above this deliquescence point, resulting in the formation of a solution.
Hydrate formation:
From the course of the sorption isotherm, conclusions in terms of possible existing hydrate phases can be derived. Hydrate formation usually manifests as a defined hysteresis between adsorption and desorption segments. Also, from the water levels detected upon these hydrate formation processes, conclusions in terms of the possible stoichiometry of the hydrate can be drawn.

Although these parameters allow a quantitative comparison of simple water uptake levels, this does not provide well-defined information on surface properties, which are essential to understanding the physical properties of materials. Also, typical data from traditional GVS experiments do not combine well with other physicochemical data from complementary methods to provide an overall picture.

It was found by the inventors that the evaluation methods according to the present invention yield meaningful physical data that correlate to relevant parameters such as dissolution rate and wettability of powders.

Accordingly, the present invention relates to the novel use of GVS for the assessment and characterisation of surface properties of particles, which particles are preferably APIs.

For this purpose, water vapour sorption experiments can be carried out as follows:
Adsorption segment range 0-98% RH, preferably 0-50% RH.
Adsorption segment RH-step size of 10% RH or smaller, preferably 5% RH or 2.5% RH.
Desorption segment range 98-0% RH, preferably 50-0% RH.
Desorption segment RH-step size of 10% RH or smaller, preferably 5% RH or 2.5% RH.
Equilibration times at each humidity of 15 minutes and longer, preferably 30 minutes and longer, most preferably 60 minutes and longer. The important factor here is that all water vapour sorption uptakes used for further evaluation do reach equilibrium, i.e. that the weight change over time within each RH-step kinetic should be very small at the end of each RH segment. For these purposes, typical equilibration times are given.
Isothermal temperature in the range 5-50° C., preferably 25-37° C., most preferably about 25° C.
Sample masses of 10-50 mg, preferably 15-50 mg. The use of a mesh sample pan as opposed to a glass crucible was shown to be beneficial in order to obtain sufficient equilibration of the whole sample surface. Compression/compaction of the sample should be avoided in order to ensure that there are enough voids in any powder sample to allow the water vapour to penetrate.

From these experiments, the primary information obtained is the sorption kinetic (mass vs. time at constant humidity) as well as the conventional sorption isotherm (weight change related to dry value vs. RH level), as previously described.

Gas sorption models used to convert the GVS sorption data to actual water surface area data may comprise the BET Model or the Excess Surface Work Model.

### BET Model

The multilayer sorption model according to Brunauer, Emmet and Teller Journal of the American Chemical Society, vol. 60, pp. 309, 1938) is described as follows:

$$\begin{matrix}\frac{n}{{n}_{mono}} = \frac{C \cdot \frac{p}{{p}_{s}}}{( 1 - \frac{p}{{p}_{s}} ) \cdot [ 1 + ( C - 1 ) \cdot \frac{p}{{p}_{s}} ]} & ( A 1 \text{-} a ) \\ ⇔ \frac{\frac{p}{{p}_{s}}}{( 1 - \frac{p}{{p}_{s}} ) \cdot n} = \frac{1}{C \cdot {n}_{mono}} \cdot [ 1 + ( C - 1 ) \cdot \frac{p}{{p}_{s}} ] & ( A 1 \text{-} b )\end{matrix}$$

with: n=number of moles adsorbed at p/p, (=RH/100)
nₘₒₙₒ=apparent number of moles adsorbed in monolayer
p/pₛ=gas partial pressure (=RH/100 in the GVS experiments)
C=BET constant (measure for the strength of the sorption interaction)

From the sorption kinetics, the equilibrium weight changes at the end of each RH stage are extracted to compile xy-data pairs (RH vs. adsorbed amount). This data corresponds to the following parameters in the BET equation (A1-b):
p/pₛ=water partial pressure, i.e. RH/100 in the GVS experiment
n=adsorbed amount, i.e. the detected weight change from the GVS experiment at each RH converted into the number of moles of water

From this, a plot of [p/pₛ]/[n(1−p/pₛ)] versus p/pₛ should give a linear correlation with intercept b=1/[C(nₘₒₙₒ)] and slope m=b(C−1).

Evaluation is done by using 5-7 data points (RH vs. adsorbed amount) in the RH range 5-30% RH, which are then used for linear regression to obtain the intercept b and slope m (as explained above). The data is considered acceptable, if the correlation coefficient (r²) of the linear regression is >0.95, although a correlation coefficient of ≧0.99 should be obtainable for most data sets. From slope and intercept, one can calculate the BET constant C and apparent monolayer amount nₘₒₙₒ.

### Excess Surface Work Model

The xy-data pairs used for the sorption isotherms (m_{RH}, RH-levels) can also be used for a different approach to describe the sorption isotherm. Here, the change in chemical potential Δμ for the sorption process at each relative humidity (and at temperature T) is calculated:

$$Δ μ = RT \cdot ln ( \frac{p}{{p}_{s}} ) = RT \cdot ln ( \frac{rh}{100} ) \qquad ( A 2 )$$

with: R=Universal Gas Constant
T=Temperature
p=water partial pressure at humidity RH and temperature T
pₛ=water vapour saturation pressure at temperature T

Δμ represents the change in chemical potential of the sorption process, since in equilibrium the chemical potential for the vapour phase equals the chemical potential of the adsorbed phase.

Based on the change in chemical potential Δμ, an energetic term φ can be calculated for each relative humidity, termed excess surface work (ESW) according to J. Adolphs and M. J. Setzer, Journal of Colloid and Interface Science, vol. 180, pp. 70-76, 1996:

Φ=n_{ads}·Δμ (A3)

with: Φ=excess surface work
Δμ=change in chemical potential
n_{ads}=number of moles of water adsorbed in equilibrium at humidity RH

A plot of Φ/RT vs. n_{ads} can then be generated, to allow the value of n_{ads} at the minimum value of Φ/RT to be deduced. This value of n_{ads} corresponds to nₘₒₙₒ.

Application of these gas sorption models for water vapour sorption data yielded the apparent monolayer coverage nₘₒₙₒ for water vapour sorption processes, and further on, the novel water surface area parameter that is one of the objects of the invention described herein.

### Water Surface Area

The calculated monolayer coverage nₘₒₙₒ obtained from the application of the gas sorption models to GVS data can be re-calculated to water surface area A_{water}, taking into account the mean cross sectional area a_{H2O} occupied by one water molecule upon adsorption:

$${A}_{water} = \frac{{a}_{{H}_{2} O} \cdot {n}_{mono} \cdot {N}_{L}}{{m}_{dry} ( sample )} \qquad ( A 4 )$$

with: A_{water}=water surface area [m²/g]
a_{H2O} mean cross sectional area of one water molecule adsorbed 10⁻¹⁹ m²/molecule
nₘₒₙₒ=apparent amount adsorbed in the monolayer [mol]
N_{L}=Lochschmidt number=6×10²³ molecules/mol
m_{dry} (sample)=dry mass of sample [g]

As an alternative, a non-gravimetric based sorption technique can be used to calculate the WSA. For example, a water partial pressure monitoring sorption system (such as that available from Quantachrome Instruments) may be used. In such a case the experiment is nearly the same, but as opposed to measuring the adsorbed amounts gravimetrically (via an ultra-microbalance), the adsorbed amounts are measured via water partial pressure measurements in defined cell volumes.

The present invention therefore also relates to the calculation and use of the described physical meaningful advanced characterisation data of water sorption processes to compare water vapour sorption data of samples.

The present invention also comprises the application of these evaluations to pharmaceutical samples, such as APIs, excipients, and compositions. Water surface area data were shown to correlate with most relevant key performance parameters, for example, dissolution rate of API powders. These data are very important, since classical characterisation techniques such as XRPD, DSC, and IR refer to physical bulk properties, which are often identical in batches with different dissolution profiles and/or bioavailability.

The different aspects of the present invention are illustrated below by a set of non-limiting examples.

### EXAMPLES

The bulk properties of four samples, A-D, of irbesartan were tested.

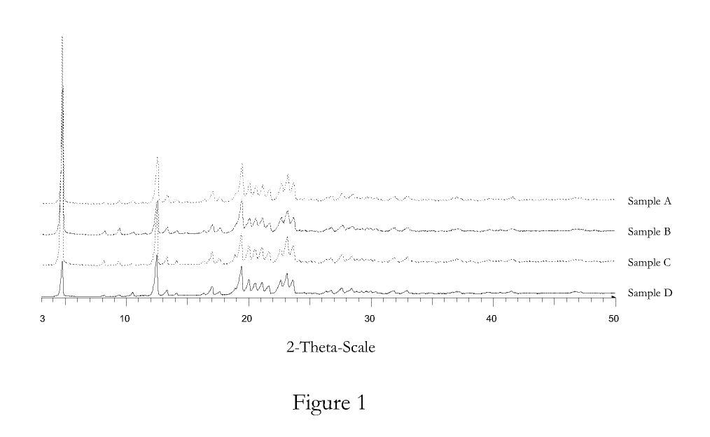

All four samples were of the same crystalline form by XRPD analysis (see FIG. 1).

The particle size profile of samples A-D of irbesartan were tested (see Table 1). It can be seen that across the range the particle sizes were very similar. Thus any difference in dissolution kinetics of samples from the relevant batch cannot be attributed to particle size.

TABLE 1
Particle size of irbesartan samples A-D.

| | A | B | C | D |
|---|---|---|---|---|
| D (v, 0.1) | 1 μm | 1 μm | 1 μm | 1 μm |
| D (v, 0.5) | 3 μm | 3 μm | 5 μm | 3 μm |
| D (v, 0.9) | 12 μm | 17 μm | 13 μm | 7 μm |

The D values represent particle size medians from the detected particle size distribution curves. D(v, x) means that 100x % of particles are smaller than the corresponding D value, and 100-100x % of particles are larger. The samples were also subjected to the Brunauer-Emmett-Teller (BET)-nitrogen adsorption analysis to determine the total surface area of the samples as follows:

Approximately 0.2-0.5 g of material (accurately weighed) was loaded into a surface area analyser glass tube fitted with a glass rod in order to minimise the overall free space. The sample was then outgased under vacuum at 30° C. for several hours. Upon completion of the outgas step, the sample tube was removed from the analyser and backfilled with helium to avoid refilling with air from the surrounding environment. The sample tube was reweighed in order to calculate the amount of dry sample used during the analysis step. The analyser tube was then loaded into the surface area analyser, and free space analysis was then conducted in order to account for the free volume in the tube and thus accurately measure the quantity of gas adsorbed directly on the powder surface. Nitrogen gas adsorption measurement was executed in a liquid nitrogen filled dewar at 77.3K. BET multipoint surface area determination was performed in the partial pressure range p/pₒ=0.1-0.3 with 5 data points. Specific surface area calculation was performed by linear regression analysis of data according to the BET equation.

The results of this determination are shown in Table 2. Sample B, which in fact had a significantly poorer dissolution profile than samples A and D, had the greatest specific surface area. Hence, it can be seen that the different dissolution behaviour of the samples is not merely a consequence of detected differences in total surface areas.

TABLE 2
Total specific surface area values of irbesartan samples
A-D calculated using the BET nitrogen adsorption model.

| Sample | Specific Surface Area (nitrogen) |
|---|---|
| Sample A | 5.17 m²/g |
| Sample B | 6.16 m²/g |
| Sample C | 3.16 m²/g |
| Sample D | 4.29 m²/g |

The four samples A-D of irbesartan were also subjected to GVS to assess the extent of any water adsorption. It was surprisingly found that samples A and D exhibited greater water vapour adsorption than samples B or C. The inventors found a correlation between the increased water vapour adsorption exhibited by samples A and D and desirable improved dissolution kinetics compared to samples B and C. The water adsorption results obtained from the GVS analysis are illustrated in Table 3.

TABLE 3
Water adsorption levels of samples A-D at
relative humidities between 0 and 90%.

Water Adsorption

| % RH | A | B | C | D |
|---|---|---|---|---|
| 5 | 0.06 wt % | 0.03 wt % | 0.04 wt % | 0.07 wt % |
| 10 | 0.09 wt % | 0.04 wt % | 0.07 wt % | 0.10 wt % |
| 15 | 0.12 wt % | 0.06 wt % | 0.08 wt % | 0.13 wt % |
| 20 | 0.14 wt % | 0.07 wt % | 0.09 wt % | 0.16 wt % |
| 25 | 0.17 wt % | 0.09 wt % | 0.11 wt % | 0.19 wt % |
| 30 | 0.20 wt % | 0.10 wt % | 0.13 wt % | n.d. |
| 35 | 0.22 wt % | 0.11 wt % | 0.14 wt % | 0.23 wt % |
| 40 | 0.24 wt % | 0.13 wt % | 0.15 wt % | 0.26 wt % |
| 50 | 0.29 wt % | 0.15 wt % | 0.18 wt % | 0.33 wt % |
| 60 | 0.34 wt % | 0.18 wt % | 0.21 wt % | 0.39 wt % |
| 70 | 0.39 wt % | 0.20 wt % | 0.23 wt % | 0.46 wt % |
| 80 | 0.44 wt % | 0.23 wt % | 0.27 wt % | 0.52 wt % |
| 90 | 0.52 wt % | 0.28 wt % | 0.31 wt % | 0.63 wt % |

In addition, surprisingly, it has been found that the water surface area of the samples can be determined by conversion of the results from the water vapour sorption analysis using common gas sorption models on the water vapour sorption data to obtain the characterisation data.

The determination of the BET water surface area is illustrated for sample A as follows. From the relative humidity data and the mass difference, p/pₛ and [p/pₛ]/[n(1−p/pₛ)] were calculated to give the values shown in Table 4.

TABLE 4
Results of the BET calculations for sample A.

| | | Rel. adsorbed | Abs. adsorbed | |
|---|---|---|---|---|
| RH (%) | p/pₛ | amount n (wt %) | amount n (mg) | [p/pₛ]/[n(1 − p/p_{ₛ})] |
| 4.98 | 0.05 | 0.0592 | 0.0110 | 4.764547177 |
| 10.01 | 0.10 | 0.0894 | 0.0166 | 6.700878411 |
| 15.00 | 0.15 | 0.1179 | 0.0219 | 8.058017728 |
| 19.99 | 0.20 | 0.1427 | 0.0265 | 9.428066775 |
| 25.02 | 0.25 | 0.1723 | 0.0320 | 10.42778074 |
| 30.00 | 0.30 | 0.1987 | 0.0369 | 11.61440186 |

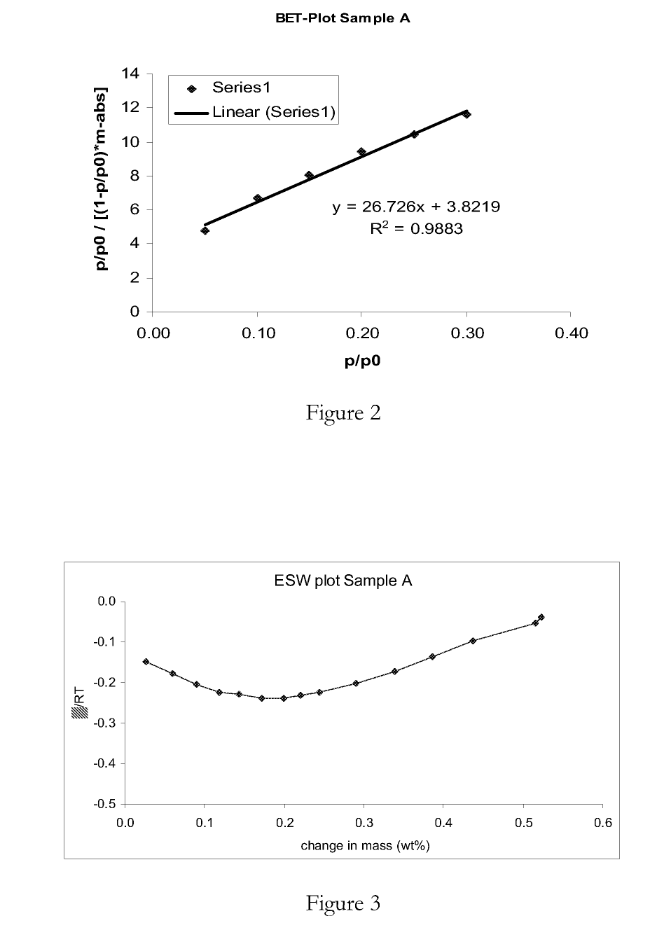

A plot of p/pₛ versus [p/pₛ]/[n(1−p/pₛ)] was then generated (see FIG. 2) allowing the slope (m=26.726) and the intercept (b=3.8219) to be calculated. Using the equations b=1/[C(nₘₒₙₒ)] and m=b(C−1), the apparent monolayer amount, n^{mono}, was calculated to equate to 0.0327 mg. This in turn led to a WSA of 5.9 m²/g, using equation (A4) above, wherein m_{dry}=18.57 mg. Similar calculations were performed on samples B-D to give the results for the BET adsorption model shown in Table 6.

Likewise, the determination of the ESW water surface area is illustrated for sample A as follows. For each relative humidity value, Δμ/RT and hence Φ/RT was calculated using equations (A2) and (A3) above. The results are shown in Table 5.

TABLE 5
Results of the ESW calculations for sample A.

| | Rel. adsorbed | | |
|---|---|---|---|
| RH (%) | amount n (wt %) | Δμ/RT = ln(RH/100) | Φ/RT |
| 0.33 | 0.0258 | −5.713833 | −0.14766 |
| 4.98 | 0.0592 | −2.99974 | −0.17765 |
| 10.01 | 0.0894 | −2.301586 | −0.20569 |
| 15.00 | 0.1179 | −1.89712 | −0.22368 |
| 19.99 | 0.1427 | −1.609938 | −0.22969 |
| 25.02 | 0.1723 | −1.385495 | −0.23869 |
| 30.00 | 0.1987 | −1.203973 | −0.23918 |
| 34.98 | 0.2207 | −1.050394 | −0.23186 |
| 40.02 | 0.2439 | −0.915791 | −0.22335 |
| 49.98 | 0.2902 | −0.693547 | −0.20126 |
| 60.00 | 0.3392 | −0.510826 | −0.17326 |
| 70.02 | 0.3855 | −0.356389 | −0.13738 |
| 79.99 | 0.4372 | −0.223269 | −0.0976 |
| 90.01 | 0.5152 | −0.105249 | −0.05423 |
| 93.03 | 0.5233 | −0.072248 | −0.03781 |

A plot of Φ/RT versus the change in mass for sample A was then generated (see FIG. 3). From this, it could be deduced that the minimum value of Φ/RT corresponded to a change in mass of 0.0369 mg, the apparent monolayer amount. This is turn equated to a WSA of 6.65 m²/g, using equation (A4) above, wherein m_{dry}=18.57 mg. Similar calculations were performed on samples B-D to give the results for the ESW adsorption model shown in Table 6.

It can be seen, in Table 6, that samples A and D have the largest water surface areas when compared to samples B or C. Based on the theory underlying the present invention, it can be predicted from the WSA data that the samples fall into two distinct groups, i.e. the relatively hydrophilic group comprising samples A and D, and the relatively hydrophobic group comprising samples B and C.

TABLE 6
Water Surface Areas of samples A-D as
obtained from GVS adsorption runs.

| Adsorption | Samples | | | |
|---|---|---|---|---|
| Model | A | B | C | D |
| ESW | 6.7 m²/g | 3.0 m²/g | 4.3 m²/g | 6.5 m²/g |
| BET | 5.9 m²/g | 3.2 m²/g | 3.6 m²/g | 6.5 m²/g |

It should also be noted that in some cases the WSA is greater than the SSA (e.g. for A, C and D). Without wishing to be bound by theory, it is believed that this is due to the fact that the WSA techniques outlined above deduce the apparent, rather than the true monolayer amount. Adsorbents such as water exhibit far greater intermolecular interactions than non-polar molecules such as nitrogen. It is therefore thought that the apparent monolayer amount for such adsorbents may often be greater than the true monolayer amount due to some of the adsorbent attaching initially in the form of clusters (i.e. as molecular aggregates).

Absorption (i.e. penetration into the bulk domains) can be ruled out for the irbesartan samples investigated, as the full adsorption-desorption isotherms did not show a broad hysteresis, with higher desorption levels throughout, which is typically observed for absorption processes.

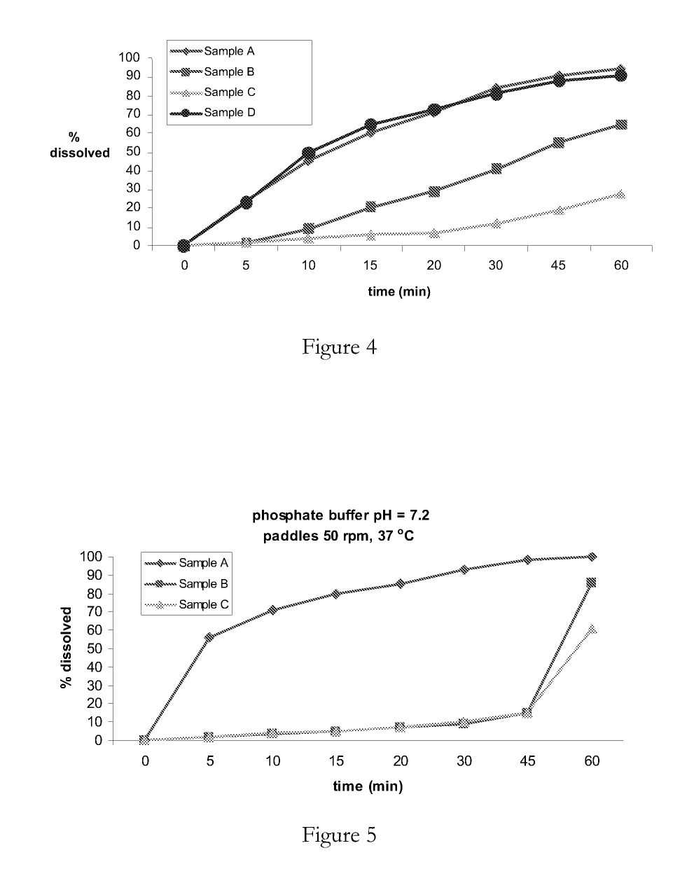

Dissolution experiments were executed in 0.1N HCt (37° C.) on the four micronised samples A-D of irbesartan. As illustrated in FIG. 4, irbesartan samples A-D, which have the same crystalline form and particle size, surprisingly have different dissolution profiles. These results were indeed predicted by the results of Table 6 showing that samples A and D have the greater water surface area.

As illustrated in FIG. 4, irbesartan samples A and D show enhanced dissolution characteristics when compared with samples B and C. Thus with all other parameters being equal, it can be seen that the improved dissolution profile directly correlates to the increased water surface area of samples A and D.

The inventors have also found that this trend is repeated over the pH range, for example up to pH 7.2 and above. The dissolution profiles of samples A-C in phosphate buffer at pH 7.2 (37° C.) are shown in FIG. 5, confirming this finding.

Not wishing to be bound by theory, it is thought that the surfaces of samples A and D possess a significantly greater number of hydrophilic domains and consequently a greater extent in hydrophilicity. Taking into account the calculated water surface areas from both the ESW model and the BET model, as well as considering the data from the GVS results, it can be said that samples A and D exhibit approximately twice the number of hydrophilic domains than samples B and C which in turn exhibit approximately a similar number of hydrophilic domains as each other.

The results observed for the calculated extent of hydrophilic domains do indeed show a correlation to the dissolution experiment results. As shown in FIGS. 4 and 5, the dissolution experiments revealed that samples A and D exhibited significantly faster dissolution kinetics in the media.

Thus, the WSA concept can be understood as an easy and quick testing method to differentiate between surface hydrophobic and surface hydrophilic samples, and hence predict which samples will show a generally good and which will show a generally bad dissolution profile.

The larger surface hydrophilicity of samples A and D as indicated by the increased water vapour sorption and water surface area shown in Table 3 and Table 6 respectively is a key factor for the dissolution behaviour in aqueous media, as the wetting of these samples is expected to be significantly better.

Consequently when formulating irbesartan into pharmaceutical compositions, batches of API with water surface areas greater than 5 m²/g are chosen to ensure consistent dissolution and bioavailability between the batches.

Experimentally observed phenomena such as dissolution rates, rate of hydrate formation and stability data clearly depend on many different factors. Thus, the present invention also relates to the use of the WSA data as an input in part of a more complex model to predict another characteristic of a substance.

For instance, the present invention also relates to a combination of water surface area data obtained by the previously described approach of the GVS data, and specific surface area data as obtained by the BET nitrogen adsorption method. In one embodiment according to the present invention, the surface area data obtained from water sorption experiments and nitrogen sorption experiments are considered as complementary data which contain different information about the surface properties of a sample:
Specific surface area (nitrogen adsorption):
The specific surface area describes the total surface geometry available for potential gas adsorption. This term however does not address surface differences in terms of their physicochemical or structural nature.
Water surface area (water vapour sorption):
The water surface area as encompassed by the present invention, can be considered as strongly selective for surface hydrophilic domains, and therefore does differentiate between surface hydrophilic and hydrophobic domains.

Further, the present invention also relates to the combination of water surface area and specific surface area (BET nitrogen) to assess surface hydrophilicity. More specifically, the ratio of water surface area to specific surface area (BET nitrogen) can be calculated to assess a dimensionless figure that allows characterisation and comparison in terms of hydrophilic domains being present in investigated samples.

Alternatively, the model may simply evaluate the lower of the WSA and the SSA. Such a value may also be taken as being predictive of the dissolution rate, since it is envisaged in some instances that where the WSA is greater than the SSA, the actual surface area of the particles becomes the rate-limiting factor rather than the extent of the hydrophilic domains. Ideally both the WSA and the SSA will be calculated using the same surface area model, preferably the BET model.

Also, applying these advanced characterisation data to adsorption and desorption data as well as on data from multiple sorption cycles allows physical meaningful conclusions in terms of possible changes of sample surface properties as a consequence to humidity exposure (humidity stressing). These changes may comprise humidity-induced surface re-arrangement processes such as crystallisation of surface-amorphous domains.

Active pharmaceutical ingredients such as those having poor aqueous solubility or poor solubility in polar non-aqueous solvents, contemplated for use in the practice of the present invention include therapeutic agents, diagnostic agents, agents of nutritional value, and the like. Examples of therapeutic agents include: analgesics/antipyretics, anaesthetics, antiasthmatics, antibiotics, antidepressants, antidiabetics, antifungal agents, antihypertensive agents, anti-inflammatories, antineoplastics, antianxiety agents, immunosuppressive agents, antimigraine agents, sedatives, antianginal agents, antipsychotic agents, antimanic agents, antiarrhythmics, antiarthritic agents, antigout agents, anticoagulants, thrombolytic agents, antifibrinolytic agents, hemorheologic agents, antiplatelet agents, anticonvulsants, anti-Parkinson agents, antihistamines/antipruritics, agents useful for calcium regulation, antibacterial agents, antiviral agents, antimicrobials, anti-infectives, bronchodilators, hormones, hypoglycemic agents, hypolipidemic agents, antiulcer/antireflux agents, antinauseants/antiemetics, and oil-soluble vitamins (e.g. vitamins A, D, E, K, and the like).

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1-78. (canceled)

79. A process for the preparation of a pharmaceutical composition comprising one or more active pharmaceutical ingredient(s) [API(s)] and one or more pharmaceutically acceptable excipient(s), comprising the steps of:
(i) selecting at least one API according to a predetermined water surface area, and
(ii) formulating the at least one API with the pharmaceutically acceptable excipient(s).

80. A process according to claim 79, wherein:
(a) the API is poorly soluble in an aqueous medium; or
(b) the API is poorly soluble in a non-aqueous medium; or
(c) the API is irbesartan; or
(d) the API is irbesartan, and wherein the water surface area is equal to or greater than about 5 m²/g.

81. A pharmaceutical composition comprising an API having a predetermined water surface area.

82. A composition according to claim 81, wherein:
(a) the composition is a solid composition; or
(b) the composition is a solid composition in the form of a tablet, a capsule, or a dry powder; or
(c) the composition is a solid composition in the form of a tablet, a capsule, or a dry powder, and wherein the API is irbesartan; or
(d) the composition is a solid composition in the form of a tablet, a capsule, or a dry powder, wherein the API is irbesartan, and wherein the water surface area is equal to or greater than about 5 m²/g; or
(e) the composition is a liquid composition; or
(f) the composition is a liquid parenteral composition; or
(g) the composition is an oral liquid; or
(h) the composition is a topical composition; or
(i) the composition is a topical composition in the form of a gel, an ointment, a balm, a nasal spray, eye drops, or a cream; or
(j) the composition is formulated for inhalation; or
(k) the composition is formulated for inhalation using a dry powder inhaler (DPI), a metered dose inhaler (MDI), or a nebule; or
(l) the composition is formulated for inhalation, and wherein the API is formoterol; or
(m) the composition is formulated for inhalation, and wherein the composition further comprises lactose.

83. A process for the preparation of pharmaceutical compositions which possess uniform dissolution and/or bioavailability, comprising the steps of:
(i) taking a sample from the or each of one or more batches of API,
(ii) measuring the water surface area of the or each sample,
(iii) selecting the batch(es) of API to be used in the preparation of pharmaceutical compositions based on the measured water surface area values, and
(iv) formulating said API from the selected batch(es) into pharmaceutical compositions.

84. A process according to claim 83, wherein:
(a) the water surface area of a sample is measured using gravimetric vapour sorption (GVS); or
(b) the batches are selected by comparing the measured water surface area values with predetermined water surface area values and selecting those batches having water surface area values within the predetermined range; or
(c) the API is poorly soluble in an aqueous medium or in a non-aqueous medium.

85. A process according to claim 83, wherein:
(a) the composition is a solid composition; or
(b) the composition is a solid composition in the form of a tablet, a capsule or a dry powder; or
(c) the composition is a solid composition in the form of a tablet, a capsule or a dry powder, and wherein the API is irbesartan; or
(d) the composition is a solid composition in the form of a tablet, a capsule or a dry powder, wherein the API is irbesartan, and wherein the water surface area is equal to or greater than about 5 m²/g; or
(e) the composition is a liquid composition; or
(f) the composition is a liquid parenteral composition; or
(g) the composition is an oral liquid; or
(h) the composition is a topical composition; or
(i) the composition is a topical composition in the form of a gel, an ointment, a balm, a nasal spray, eye drops, or a cream; or
(j) the composition is formulated for use in an inhaler; or
(k) the composition is formulated for use in a dry powder inhaler (DPI), a metered dose inhaler (MDI), or a nebule; or
(l) the composition is formulated for use in an inhaler, and wherein the API is formoterol; or
(m) the composition is formulated for use in an inhaler, and wherein the composition further comprises lactose.

86. A method for determining the water surface area of an API, comprising the steps of:
(i) measuring water vapour sorption isotherms of a sample of the API, and
(ii) applying a model for determining the water surface area.

87. A method according to claim 86, wherein:
(a) the isotherms are measured using gravimetric vapour sorption (GVS); or
(b) the model is the Excess Surface Work (ESW) model or the Brunauer, Emmet and Teller (BET) model.

88. A method for determining the water surface area of a particle, comprising the steps of:
(i) measuring water vapour sorption isotherms of a sample of the particle, and
(ii) applying a model for determining the water surface area.

89. A method according to claim 88, wherein:
(a) the isotherms are measured using gravimetric vapour sorption (GVS); or
(b) the model is the Excess Surface Work (ESW) model or the Brunauer, Emmet and Teller (BET) model.

90. Irbesartan with a water surface area equal to or greater than about 5 m²/g.

91. An active pharmaceutical ingredient (API) for use in an inhaler, said API having a water surface area that allows for optimized adherence to a support suitable for use in the inhaler.

92. An API according to claim 91, wherein:
(a) the API is one or more of formoterol, salmeterol, fluticasone, budesonide, or a pharmaceutically acceptable salt thereof; or
(b) the API is one or more of salmeterol xinafoate, fluticasone propionate, budesonide, or formoterol fumarate; or
(c) the inhaler is a dry powder inhaler (DPI); or
(d) the support is a particulate support; or
(e) the support is lactose.

93. Formoterol with a water surface area that allows for optimized adherence of the formoterol to a support suitable for use in an inhaler.

94. Formoterol according to claim 93, wherein:
(a) the inhaler is a dry powder inhaler (DPI); or
(b) the support is a particulate support; or
(c) the support is lactose.

95. A process for assessing the surface hydrophilicity of an API or a particle, comprising combining water surface area and specific surface area.

96. A process comprising measuring the water surface area of a substance.

97. A process according to claim 96, wherein said measuring comprises the steps of:
(i) measuring water vapour sorption isotherms of a sample of the substance, and
(ii) applying a model for determining the water surface area.

98. A process according to claim 97, wherein:
(a) the isotherms are measured using gravimetric vapour sorption (GVS) or a water partial pressure monitoring sorption system; or
(b) the model is the Excess Surface Work (ESW) model or the Brunauer, Emmet and Teller (BET) model.

99. A process according to claim 96, wherein:
(a) the substance is poorly soluble in an aqueous medium; or
(b) the substance is poorly soluble in a non-aqueous medium; or
(c) the substance is a solid; or
(d) the substance is a particulate or a powder; or
(e) the substance is an active pharmaceutical ingredient (API); or
(f) the substance is irbesartan, formoterol, salmeterol, fluticasone, or budesonide.

100. A process according to claim 96, further comprising the step of:
(a) comparing the water surface area with a predetermined value or range of values; or
(b) using the water surface area to predict another property of the substance; or
(c) using the water surface area to predict the dissolution rate of the substance; or
(d) using the water surface area in conjunction with another value to predict another property of the substance; or
(e) using the water surface area in conjunction with the specific surface area of the substance to predict another property of the substance.

101. A method of manufacturing a substance, said method comprising the process as claimed in claim 96.

102. A method of manufacturing a substance, wherein the manufacturing process is performed to meet a water surface area value.

103. A method of altering a manufacturing process for a substance, comprising the alteration of a process variable in response to a water surface area value.
